(19) United States
(12) Patent Application Publication
(10) Pub. No.: US 2010/0217263 A1
(43) Pub. Date: Aug. 26, 2010

(54) AUTOMATED HAIR REMOVAL DEVICE

(75) Inventor: Sladjana Tukulj-Popovic, Toronto (CA)

Correspondence Address: Myers Andras Sherman LLP, 19900 MacArthur Blvd., Suite 1150, Irvine, CA, 92612, US

(73) Assignee: Thane International, Inc., La Quinta, CA (US)

(21) Appl. No.: 12/712,837

(22) Filed: Feb. 25, 2010

Related U.S. Application Data
(60) Provisional application No. 61/155,873, filed on Feb. 26, 2009.

(51) Int. Cl. A61B 17/50

(52) U.S. Cl. 606/43; 606/133

(57) ABSTRACT

An automated hair removal system is provided. The system includes a handheld device having an electric motor for rotating an attachment holder, the attachment holder including a head attachment mechanism for receiving an attachment head. The system further includes an attachment head having a contact surface configured for skin contact, the attachment head further having a holder attachment mechanism for detachably attaching the attachment head to the attachment holder. Rotation of the attachment holder by the electric motor causes rotation of the attachment head and the contact surface, and wherein rotation of the contact surface upon placement against the skin essentially in parallel to the skin and transverse to hair thereon, removes hair from the skin by tapering and weakening the structure of the hair.

### CROSS-REFERENCE TO RELATED APPLICATIONS

This application claims priority from U.S. Provisional Patent Application Ser. No. 61/155,873, filed on Feb. 26, 2009, incorporated herein by reference.

### FIELD OF THE INVENTION

The present invention relates generally to hair removal devices, and in particular, to automated hair removal devices.

### BACKGROUND OF THE INVENTION

Hair removal has been practiced in almost all human cultures. Various hair removal methods have been utilized to remove hair from different body areas for medical, social, religious, cultural, sexual, etc., reasons. In many cultures, particularly North American and Western European, it became increasingly common during the 20th century for women to remove some or all of their body hair, due to societal values that consider it unattractive and/or not feminine or as a matter of practicing good hygiene. In some religions, it is recommended to remove pubic and armpit hair as a hygienic practice.

### BRIEF SUMMARY OF THE INVENTION

The present invention provides an automated hair removal system. In one embodiment of the invention, the system includes a handheld device having an electric motor for rotating an attachment holder, the attachment holder including a head attachment mechanism for receiving an attachment head. The system further includes an attachment head having a contact surface configured for skin contact, the attachment head further having a holder attachment mechanism for detachably attaching the attachment head to the attachment holder. Rotation of the attachment holder by the electric motor causes rotation of the attachment head and the contact surface, and wherein rotation of the contact surface upon placement against the skin essentially in parallel to the skin and transverse to hair thereon, removes hair from the skin by tapering and weakening the structure of the hair.

These and other features, aspects and advantages of the present invention will become understood with reference to the following description and accompanying figures.

### BRIEF DESCRIPTION OF THE DRAWINGS

FIG. 1 shows an exploded view of an automated hair removal system, according to an embodiment of the invention.

FIG. 2A shows a front view of the automated hair removal system of FIG. 1, according to an embodiment of the invention.

FIG. 2B shows a cross-section side view of the automated hair removal system of FIG. 2A, along a long axis thereof, according to an embodiment of the invention.

FIG. 3A shows a perspective view of the automated hair removal device without an attachment head, according to an embodiment of the invention.

FIG. 3B shows examples of attachment heads for the automated hair removal device, according to an embodiment of the invention.

FIG. 3C shows a perspective view of the automated device of FIG. 3A, with an attachment head, according to an embodiment of the invention.

FIG. 3D shows a back perspective view of an example attachment head showing example protrusions for engaging an attachment head holder, according to an embodiment of the invention.

FIG. 3E shows a front view of an attachment head holder for the attachment head of FIG. 3D, according to an embodiment of the invention.

FIG. 3F shows a back perspective view of another example attachment head example protrusions for engaging an attachment head holder, according to an embodiment of the invention.

FIG. 3G shows a front view of an attachment head holder for the attachment head of FIG. 3F, according to an embodiment of the invention.

FIG. 4 shows more a detailed view of an electrical motor and transfer gears for transferring rotation of the motor shaft to an attachment head in the system of FIG. 1, according to an embodiment of the invention.

FIG. 5 shows an exploded view of an automated hair removal system, according to another embodiment of the invention.

FIG. 6A shows a front view of the automated hair removal system of FIG. 5, according to an embodiment of the invention.

FIG. 6B shows a cross-section side view of the automated hair removal system of FIG. 6A, according to an embodiment of the invention.

FIG. 7A shows a perspective view of an automated hair removal device without an attachment head, according to an embodiment of the invention.

FIG. 7B shows examples of attachment heads, according to an embodiment of the invention.

FIG. 7C shows a perspective view of the automated hair removal device of FIG. 7A, with an attachment head, according to an embodiment of the invention.

FIG. 8 shows a more detailed view of an electrical motor and transfer gears for transferring rotation of the motor shaft to an attachment head in the system of FIG. 5, according to an embodiment of the invention.

FIG. 9 shows a process in which a user utilizes an automated hair removal device for removing hair on skin, according to an embodiment of the invention.

FIGS. 10A-C shows more detailed illustrations of application of the automated hair removal device with an abrasive disc to human skin, according to an embodiment of the invention.

### DETAILED DESCRIPTION OF THE INVENTION

The following description is made for the purpose of illustrating the general principles of the invention and is not meant to limit the inventive concepts described herein. Further, particular features described herein can be used in combination with other described features in each of the various possible combinations and permutations. Unless otherwise specifically defined herein, all terms are to be given their broadest possible interpretation including meanings implied from the specification as well as meanings understood by those skilled in the art and/or as defined in dictionaries, treatises, etc. The description may disclose several preferred embodiments for an automated hair removal device. While the following description will be described in terms of such languages for clarity and placing the invention in context, it should be kept in mind that the teachings herein may have broad application to all types of systems, devices and applications.

The present invention provides an automated hair removal system which in one embodiment comprises an electrically operated hair removal device for removing hair from the body such as a human body using a rotating disc of abrasive material. In one embodiment, the electric hair removal device is shaped ergonomically to fit in a human hand.

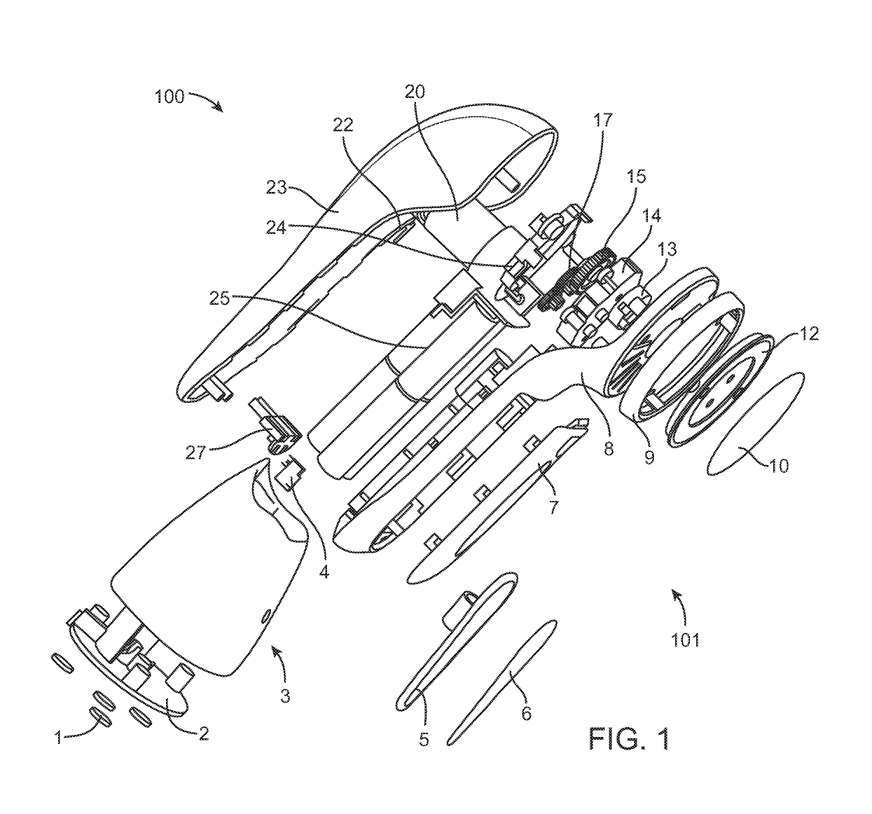

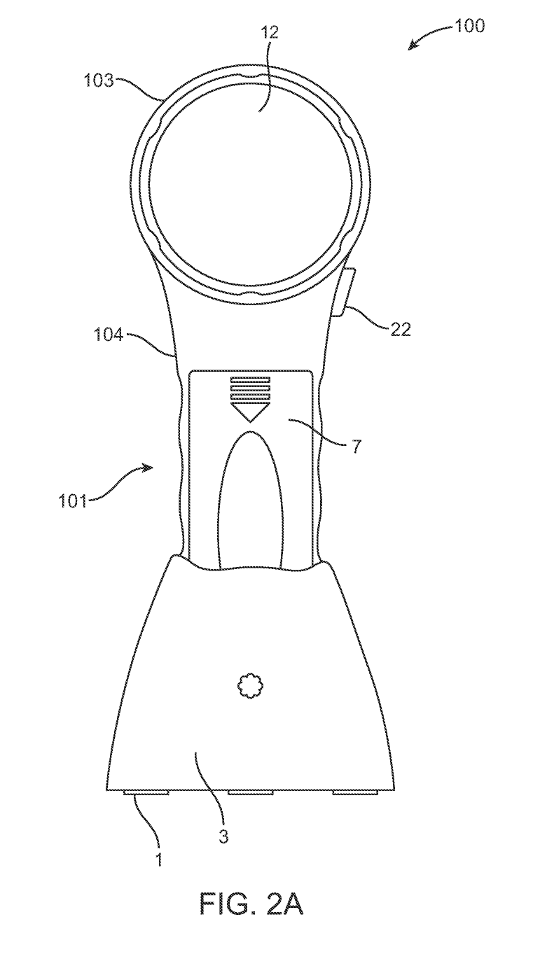

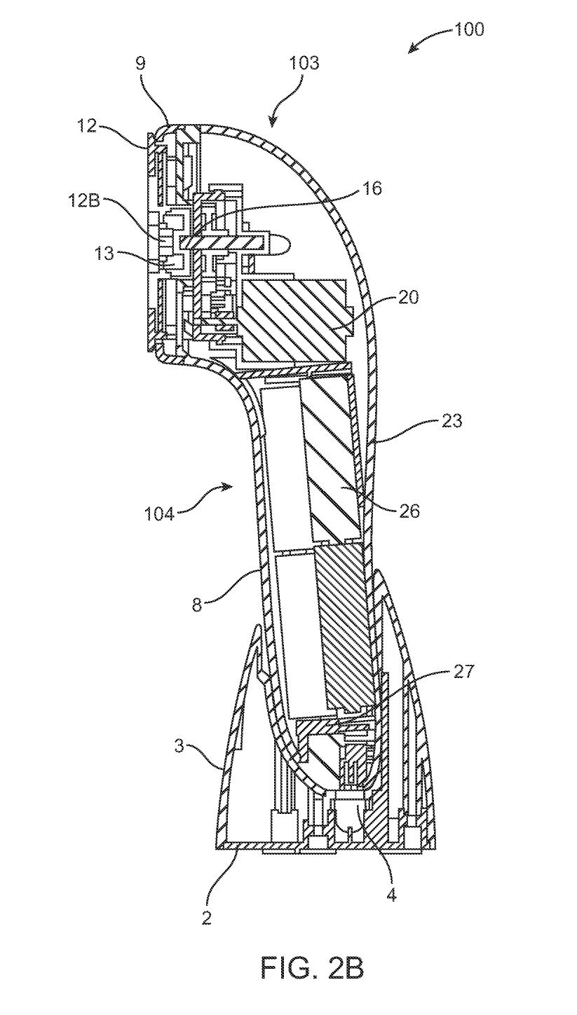

FIG. 1 shows an exploded view of a first embodiment of an automated hair removal system 100, according to an embodiment of the invention. The system 100 comprises an essentially cylindrical stand 3 with a planar base 2 having rubber feet 1 attached to an underside thereon. The system 100 further includes a hand held automated hair removal device 101. FIG. 2A shows a front view of the automated hair removal system of FIG. 1, illustrating that the stand 3 provides a standing station when the device 101 is not in use. The stand 3 is shaped to receive a bottom portion of the device 101, wherein the device 101 slides into stand 3. To use the device 101, a user simply slides the device 101 out of the stand 3.

A hand tool 5 and sand paper 6 are also provided. The hand tool 5 is a manual facial hair remover and the sand paper 6 is applied to the tool 5 for that purpose. In one implementation, the device 101 comprises a small electric motor such as a direct current (DC) electric motor 20 powered by one or more batteries 25 (e.g., 9V from six alkaline 1.5V batteries). A battery contact 27 provides electrical connection for charging the batteries 25. In one example, the electric motor may comprise a Permanent Magnet DC (PMDC) motor, no load current 0.37A max, rated speed 17000 revolutions per minute (RPM), no load starting voltage 0.8A max, rated current 2.43A max, rated voltage 9.5V.

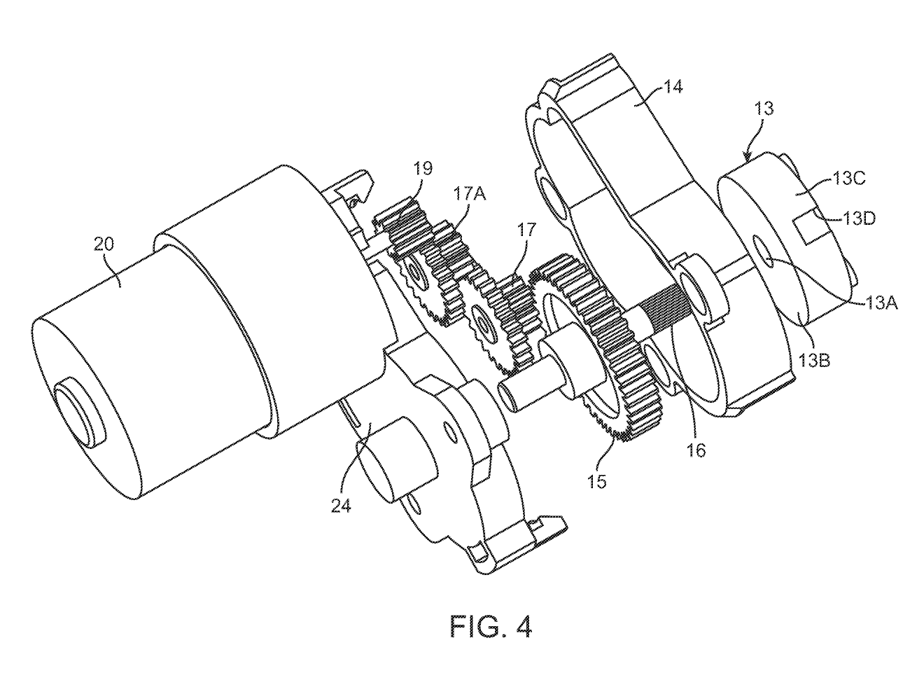

The device 101 further includes a housing comprising a back shell 23 and a corresponding front shell 8. The shells 8, 23 may be made of resilient materials such as plastics and metals. The shells 8 and 23 are interlocking, providing a handle portion 104 and a head portion 103 (FIG. 2A). FIG. 2B shows a cross-section side view of the automated hair removal system of FIG. 2A. As shown in FIGS. 1 and 2A-2B, the handle portion 104 includes a handle portion cavity for disposing the batteries 25 therein. The head portion 103 includes a head portion cavity for disposing the electric motor 20 and transfer gears 17A, 17 and 15 (FIG. 4) therein, described further below. The handle portion 104 is ergonomically shaped for being held by a human user hand. Table 1 below provides example specifications for the gears 19, 17, 17A, 15.

TABLE 1
Gear specifications

| | | | | basic | outside | Pressure |
|---|---|---|---|---|---|---|
| number | name | Z | M | diameter | diameter | angle |
| 19 | gear | 9 | 0.5 | 4.5 | 5.3 | 20 degree |
| 17A | gear | 12 and 28 | 0.5 | 6 and 14 | 6.8 and 14.8 | 20 degree |
| 17 | gear | 12 and 28 | 0.5 | 6 and 14 | 6.8 and 14.8 | 20 degree |
| 15 | gear | 44 | 0.5 | 22 | 22.8 | 20 degree |

The front shell 8 includes a battery door 7 for access to the batteries 25. The batteries 25 are placed in the handle cavity to supply DC electrical power to the motor 20. The device 101 may also include a DC power jack 4 (FIG. 1) providing an option to operate the electric motor 20 directly from an external adapter (e.g., 120V AC to 9V DC adapter), without batteries 25 or when the batteries 25 are out of charge. When the batteries 25 have sufficient charge, the device 101 may be operated as a handheld cordless unit that is powered by the batteries therein.

Electrical power to the motor 20 is activated by a push on/off switch 22. The switch 22 is placed in a circuit between the motor 20 and the power source (e.g., batteries 25, DC power jack 4) to open and close the circuit when a user closes/opens the switch.

The device 101 may include an AC to DC converter therein for converting alternating current (AC) to DC for the motor 20. The electric motor 20 may also comprise an AC electric motor energized directly from an AC line power. The on/off electrical switch 22 connects/disconnects AC or DC power source to the electric motor 20.

In one example, the device 101 is about 8 inches high with the handle portion 104 being about 1.5 inches in diameter, the head portion 103 being about 2.5 inches in diameter.

FIG. 4 shows a more detailed view of the motor 20 and the transfer gears 17A, 17 and 15. The electric motor 20 has a motor shaft gear 19 coupled to the interlocking transfer gears 17A, 17 and 15. Rotation of the motor shaft gear 19 is harnessed by said transfer gears to rotate an essentially circular attachment head holder 12. Specifically, the motor shaft gear 19 is interlocked with the reduction gear 17A, which in turn is interlocked with the reduction gear 17. The reduction gear 17 is in turn interlocked with the direct gear 15. The gear 15 includes a shaft 16 that is mechanically coupled to the attachment head holder 12. The gears 17A, 17 and 15, successively reduce the RPM transferred from the motor gear 19 to the attachment head holder 12.

As the shaft 16 is a mechanical part of the gear 15, the shaft 16 rotates with the direct gear 15. A coupler 13 is engagingly coupled to the shaft 16 to rotate therewith. The coupler 13 comprises an essentially circular planar base 13B with a central flange 13A for engagingly receiving a portion of the shaft 16. The coupler 13 further comprises walls 13C extending upwardly from the base 13B, said walls having notches 13D for engaging corresponding protrusions 12B (FIG. 2B) at the back of the attachment head holder 12. A battery bracket 26 maintains the batteries 25 in the handle portion.

The gear sizes for gears 19, 17A, 17 and 15 are selected to transfer a desired RPM from the motor 20 to the attachment head holder 12. In one example, the motor 20 and gears 19, 17A, 17 and 15 are selected such that the attachment head holder 12 rotates, for example, at about 700 to 800 RPM. A ring 9 is coupled to the attachment head holder 12 at periphery thereto, causing the ring 9 to rotate with the attachment head holder 12. FIG. 4 also shows transfer gear casing comprising top case portion 14 and bottom case portion 24. The casing maintains the gears in interlocking arrangement.

In one embodiment of the device 101, the attachment head holder 12 accepts an attachment head 10 having a contact surface for skin contact (FIG. 1). Rotation of the attachment head holder 12 and the head 10 is in plane essentially perpendicular to the axis of rotation of the motor shaft 16.

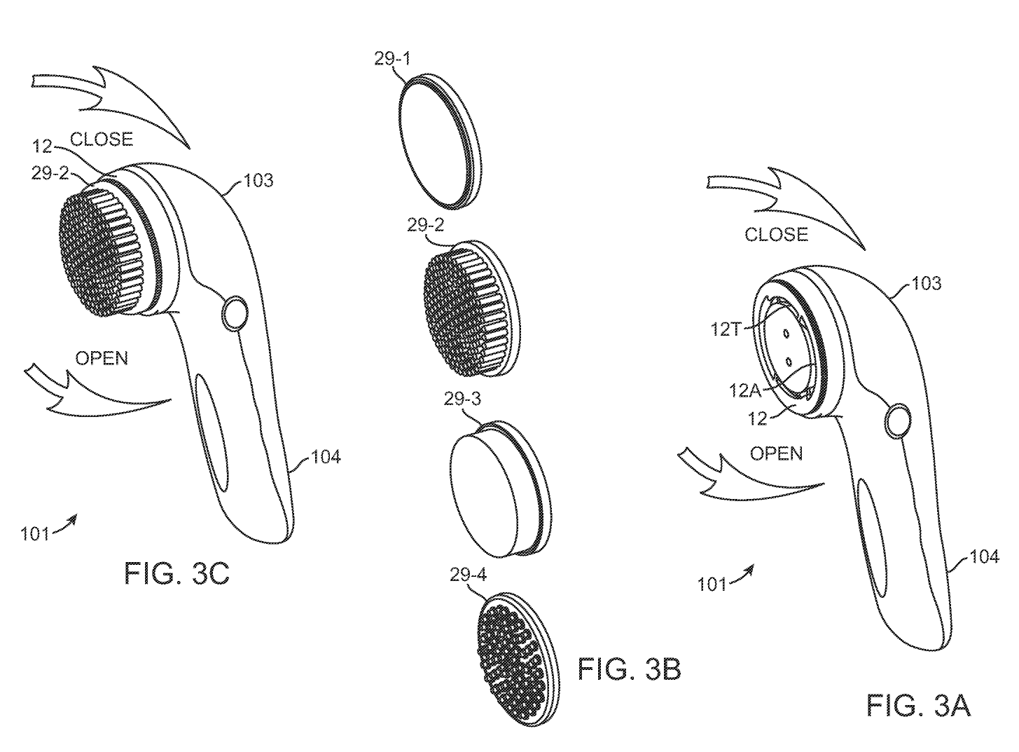

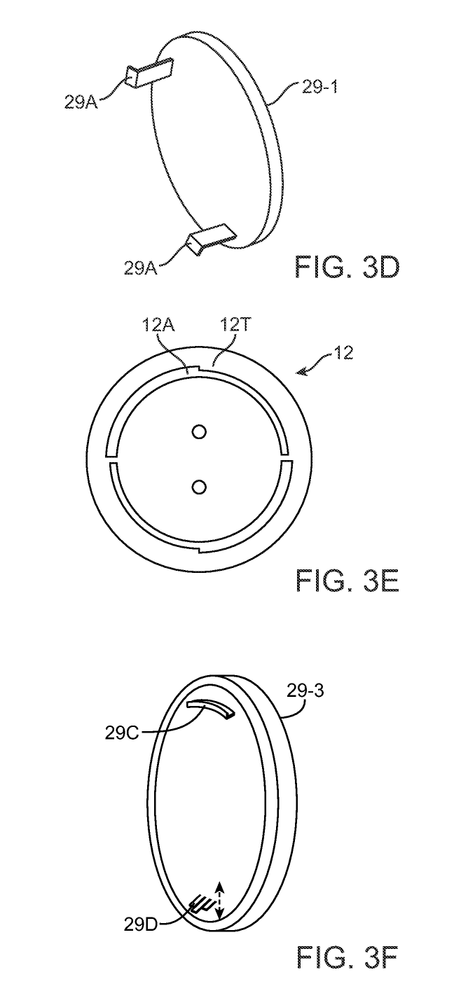

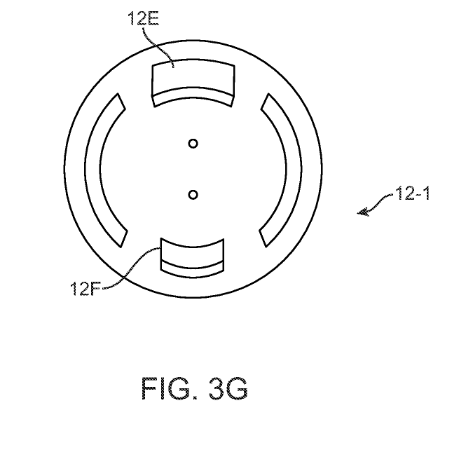

FIG. 3A shows a perspective view of the device 101, and FIG. 3B shows different example of the attachment head 10 designated as attachment heads 29-1, 29-2, 29-3 and 29-4. The attachment head holder 12 accepts different attachment heads using a twist and lock operation. The attachment head 29-1 is essentially disc shaped and has a contact surface comprising a sand paper type abrasive surface for skin contact. In one example, attachment 29-1 is a sand paper disc with grid size 1200, which is 0.33 mm thick with a self adhesive back.

The attachment head 29-2 is essentially disc shaped and has a contact surface comprising a brush head type surface for skin contact (e.g., plastic such as Acrylonitrile Butadiene Styrene for base, Nylon for brush).

The attachment head 29-3 is essentially disc shaped and has a contact surface comprising a sponge (plastic such as Acrylonitrile Butadiene Styrene for base, PVA (Polyvinyl Alcohol) for the sponge).

The attachment head 29-4 is essentially disc shaped and has a contact surface comprising a massage head type surface for skin contact (e.g., plastic such as Acrylonitrile Butadiene Styrene for base, Rubber-massaging nubs). Other attachment heads with other surfaces may be utilized with the device 101. FIG. 3C shows the device 101 with the attachment head 29-2 engaged in the holder 12. In one example, diameter of all attachment heads is 6.4 cm.

As shown in FIG. 3A, the surface of the attachment head holder 12 in the head portion 103 includes a mechanism 12A for engaging a head panel attachment 29-1 as shown by example in FIG. 3B. FIG. 3E shows a front view of an attachment head holder 12, according to an embodiment of the invention. In this example, the mechanism 12A comprises at least a channel (groove) in the surface of the attachment head holder 12 shaped and sized for engaging corresponding “L” shaped protrusions 29A at the back of the head panel attachment 29-1 shown in FIG. 3D.

FIG. 3F shows a back perspective view of another example attachment head (such as attachment head 29-3) with example protrusions 29C and 29D for engaging another example attachment head holder 12-1 illustrated in FIG. 3G, according to the invention. FIG. 3G shows the face of the attachment head holder 12-1 comprising openings 12E and 12F for receiving protrusions 29C and 29D, respectively. The opening 12F is smaller than the opening 12E.

The example attachment head 29-3 in FIG. 3F provides a clip-on mechanism in conjunction with the attachment head 12-1 in FIG. 3G. The “L” shaped protrusion 29D comprises one or more flexing spring tabs transverse to the back plane of the attachment head 29-3, wherein the tabs are bendable along the dashed arrows, to allow clipping the attachment head 29-3 to the attachment holder 12-1. To couple the attachment head 29-3 to the attachment head holder 12-1, the user holds the back plane of the attachment head 29-3 against the face of the attachment head holder 12-1, places the protrusion 29D in the opening 12F, then pushes the attachment head 29-3 against the attachment holder 12-1 (the protrusion 29D bending) until the protrusion 29C snaps into the opening 12E, and is held in place as the protrusion 29D springs back to shape in the opening 12F.

To remove the attachment head 29-3 from the attachment holder 12-1, the user grasps the periphery of the attachment head 29-3, and unclips the attachment head 29-3 from the attachment head holder 12-1 by sliding the attachment head 29-3 to bend the protrusions 29D, pulling out the protrusion 29C from the opening 12E, and pulling out the protrusion 29D from the opening 12F. Then, another attachment head may be attached to the attachment head holder 12-1. Other mechanisms for detachably attaching an attachment head to an attachment head holder may be utilized.

To couple the attachment head 29-1 to the attachment head holder 12 using a twist and lock operation, the protrusions 29A are inserted in the channel 12A by a user. The channel 12A has retaining tabs 12T such that when the attachment head 29-1 is turned clockwise relative to the attachment head holder 12, distal ends of the protrusions 29A rotate under the retaining tabs to a closed position, preventing the attachment head 29-1 from detaching from the attachment head holder 12.

To decouple the attachment head 29-1 from the attachment head holder 12 using a twist and unlock operation, the user turns the attachment head 29-1 counter clockwise relative to the attachment head holder 12, such that distal edges of the protrusions 29A rotate out from under the retaining tabs to an open position, allowing the attachment head 29-1 to detach from the attachment head holder 12.

When an attachment head with an abrasive disk 10 is coupled to the device 101 and lightly pressed to the surface of human skin, the rotation of the attachment disc 10 by the motor 20 of the device 101 smoothes and removes hair in an easy, painless and quick manner. With repeated application, hair becomes thinner and finer and removal intervals become longer.

As shown by example in FIG. 9, the invention provides a method of hair removal comprising providing an automated device 101 for automated hair removal by a rotating abrasive attachment head disc 10, such as attachment head 29-1 in FIG. 3B, wherein hair is removed by passes of the rotating disc over an area on human skin where hair is located.

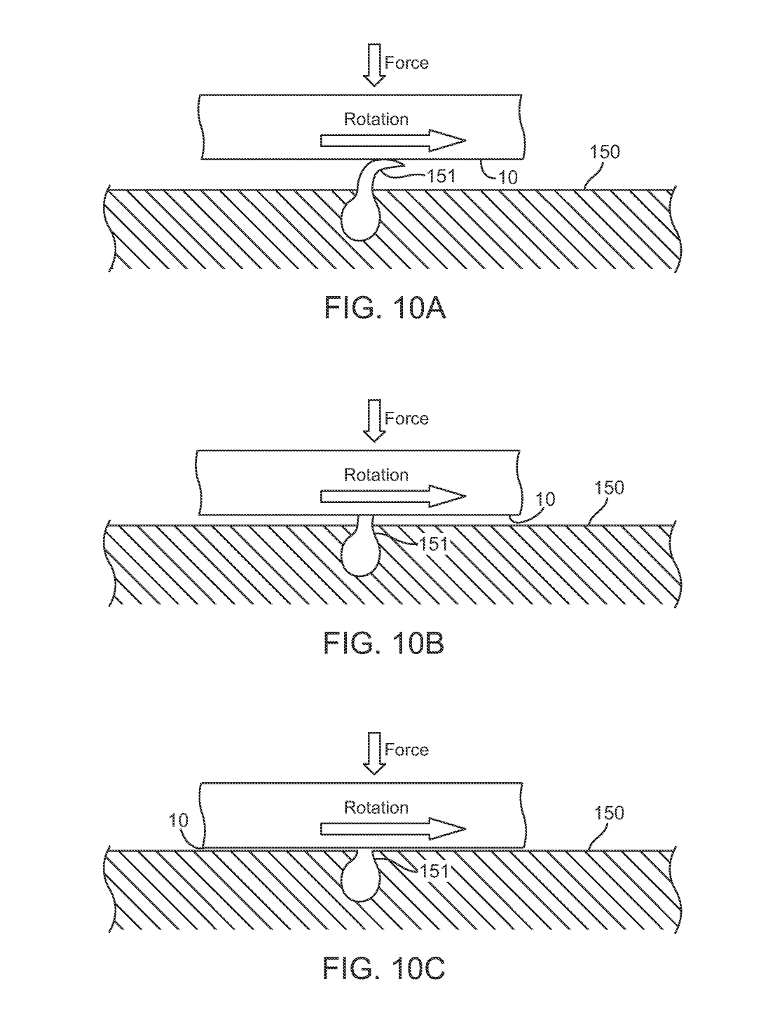

FIG. 10A shows a more detailed illustration of human skin 150 with hair 151, and a side view of contact surface 10 of an attachment head of the device 101, in initial contact with the hair 151. FIG. 10A shows direction of rotation of the contact surface 10, and direction of force on the skin 150 by the contact surface 10 as the user presses the device 101 against the skin 150. The disc 10 rotates essentially in parallel to the skin 150, wherein direction of rotation of the disc 10 is essentially transverse to the hair growth on the skin 150. FIG. 10B shows rotation of the abrasive disc 10, gradually, weakening the structure of the hair to 151. FIG. 10C shows rotation of the abrasive disc 10, gradually, weakening the structure of the hair to 151 the point where the hair 151 is completely tapered off or sanded to the root.

As such, multiple passes of the disc 10 over the same hair due to rotation of the abrasive disc 10, gradually, but rapidly, weakens the structure of the hair to the point where the hair is completely tapered off or sanded to the root. The rotation of the attachment disc leads to tapering off and sanding the hair, resulting in effective and smooth hair removal. The rotating abrasive disc provides rapid hair removal that is essentially painless and irritation free, without tiring user's hand, and can be used to access all body areas with hair.

The automated device 101 may be used with attachment heads that provide other effects in addition to or in place of hair removal. For example, the automated device 101 may be used with an attachment head providing a massaging effect (e.g., attachment head 29-4 in FIG. 3B) by automatically rotating attachment head. The process is not tiring for the hand while holding the device 101 with the rotating disc against the skin since there is no need to move the hand at a fast rate, but rather in slow motions following the area of interest on the skin.

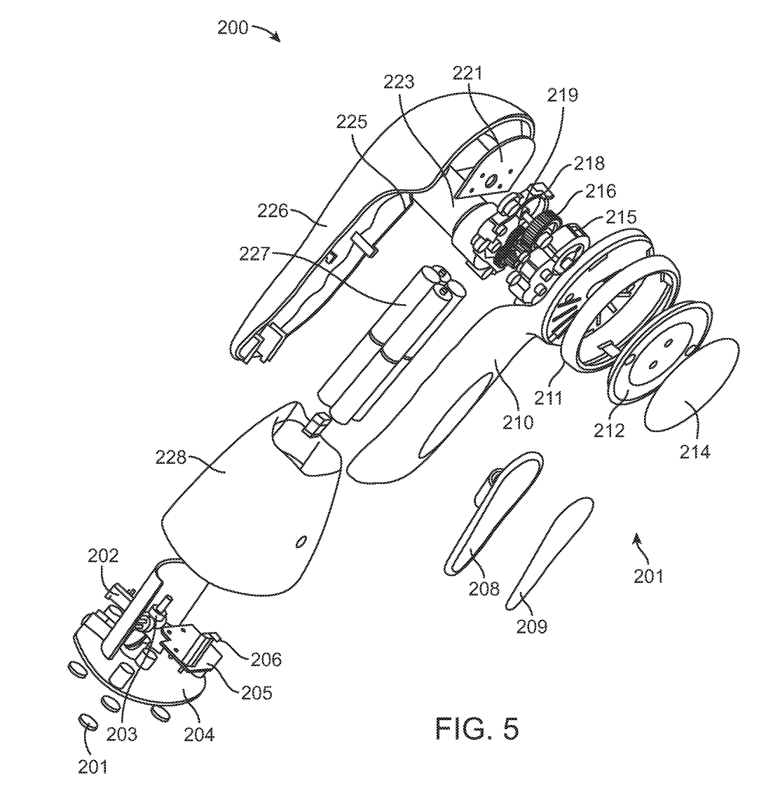

FIG. 5 shows an exploded view of an automated hair removal system 200, according to another embodiment of the invention. The hair removal system 200 (FIGS. 5-8) is mechanically similar to the hair removal system 100 (FIGS. 1-4) described above. However, instead of using disposable batteries in the hair removal system 100, the system hair removal 200 uses rechargeable batteries and includes circuitry for charging, and operating on, rechargeable batteries. As such, the mechanical aspects of the system hair removal 200 are not described in as much detail below.

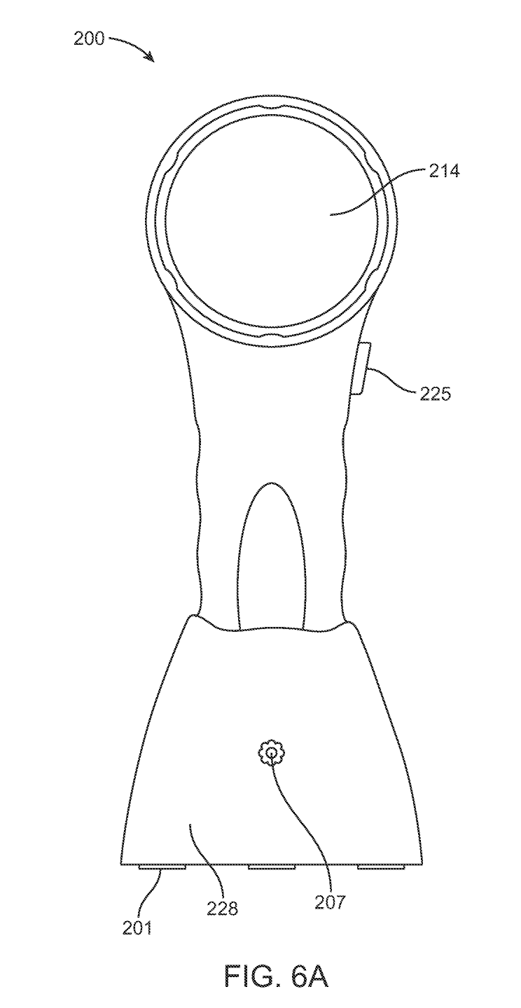

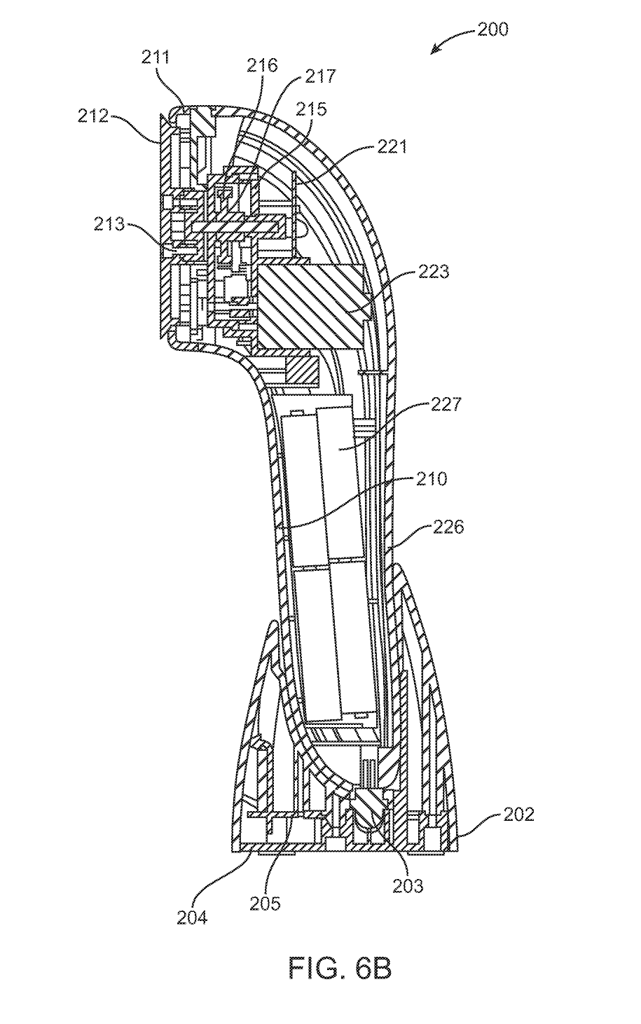

The system 200 comprises an essentially cylindrical stand 228 with a planar base 204 having rubber feet 201 attached to an underside thereon. The system 200 further includes a hand held automated hair removal device 201. FIG. 6A shows a front view of the automated hair removal system 200 of FIG. 5. The stand 228 provides a standing station when the device 201 is not in use. FIG. 6B shows a cross-section side view of the automated hair removal system of FIG. 6A. The device 201 comprises a small electric motor such as a direct current (DC) electric motor 223 powered by one or more rechargeable batteries 227. An LED stand 206 supports an LED light 207 (FIG. 6A) for indicating charge status of the batteries 227. A hand tool 208 and sand paper 209 are also provided, similar to abovementioned elements 5 and 6, respectively.

The device 201 further includes a housing comprising a back shell 226 and a corresponding front shell 210. The shells 226 and 210 may be made of resilient materials such as plastics and metals. The shells 226 and 210 are interlocking, providing a handle portion and a head portion similar to that for the device 101 in FIG. 1. The handle portion of the device 201 includes a handle portion cavity for disposing the batteries 227 therein.

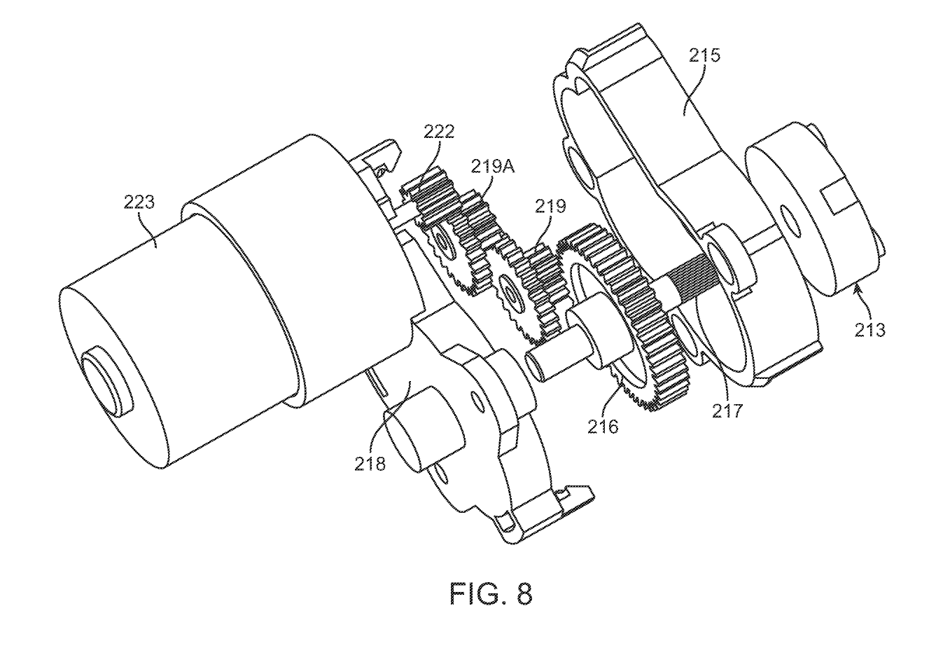

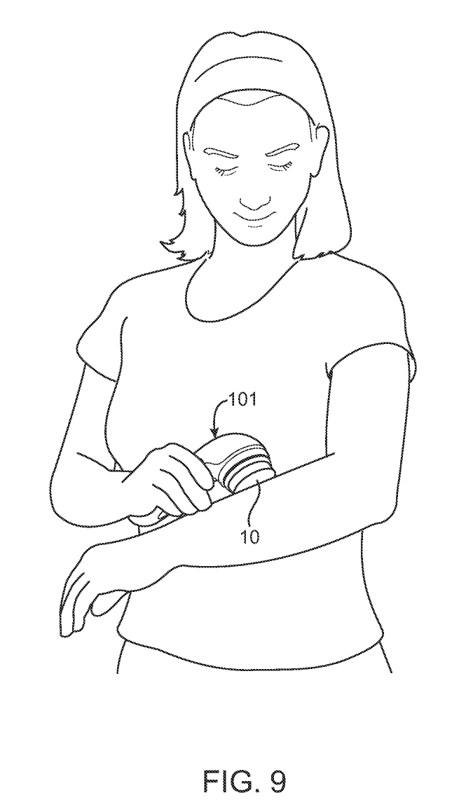

FIG. 8 shows a more detailed view of an electrical motor and transfer gears for transferring rotation of the motor shaft to an attachment head in the system of FIG. 5. The head portion of the device 201 includes a head portion cavity for disposing the electric motor 223 and transfer gears 219A, 219 and 216 therein. The handle portion is ergonomically shaped for being held by a human user hand.

Electrical power to the motor 223 is activated by a switch 225. In one example, the switch 225 comprises a sliding power switch with settings off/low/high for different motor speeds, wherein the electrical motor is a multi-speed motor. The switch 225 is placed in a circuit between the motor 223 and the electrical power source (e.g., rechargeable batteries 227) to open and close the circuit when a user closes/opens the switch 225.

The device 201 may include an AC to DC converter therein for converting AC to DC power for the motor 223. The electric motor 223 may also comprise an AC electric motor energized directly from an AC line power. The on/off electrical switch 225 connects/disconnects AC or DC power source to the electric motor 223. The device 201 may have similar dimension to device 101.

FIG. 8 shows a more detailed view of the motor 223 and the transfer gears 219A, 219 and 216. The electric motor 223 has a motor shaft gear 222 coupled to the interlocking transfer gears 219A, 219 and 216. Rotation of the motor shaft gear 222 is harnessed by said transfer gears to rotate an essentially circular attachment head holder 212. Specifically, the motor shaft gear 222 is interlocked with the reduction gear 219A, which in turn is interlocked with the reduction gear 219. The reduction gear 219 is in turn interlocked with the direct gear 216. The gear 216 includes a shaft 217 that is mechanically coupled to the attachment head holder 212. The gears 219A, 219 and 216 successively reduce the RPM transferred from the motor gear 222 to the attachment head holder 212.

As the shaft 217 is a mechanical part of the gear 216, the shaft 217 rotates with the direct gear 216. A coupler 213 is engagingly coupled to the shaft 217 to rotate therewith. The coupler 213 is also coupled to the attachment head holder 212. The coupler 213 and the attachment head holder 212 are similar to the coupler 13 and attachment head holder 12 described above in relation to the device 101, and as such are not described further.

The gear sizes for gears 222, 219A, 219 and 216 are selected to transfer a desired RPM from the motor 223 to the attachment head holder 212. In one example, the motor 223 and gears 222, 219A, 219 and 216 are selected such that the attachment head holder 212 rotates, for example, at about 700 to 800 RPM. A ring 211 is coupled to the attachment head holder 212 at periphery thereto, causing the ring 211 to rotate with the attachment head holder 212. FIG. 8 also shows transfer gear casing comprising top case portion 215 and bottom case portion 218. The casing maintains the gears in interlocking arrangement.

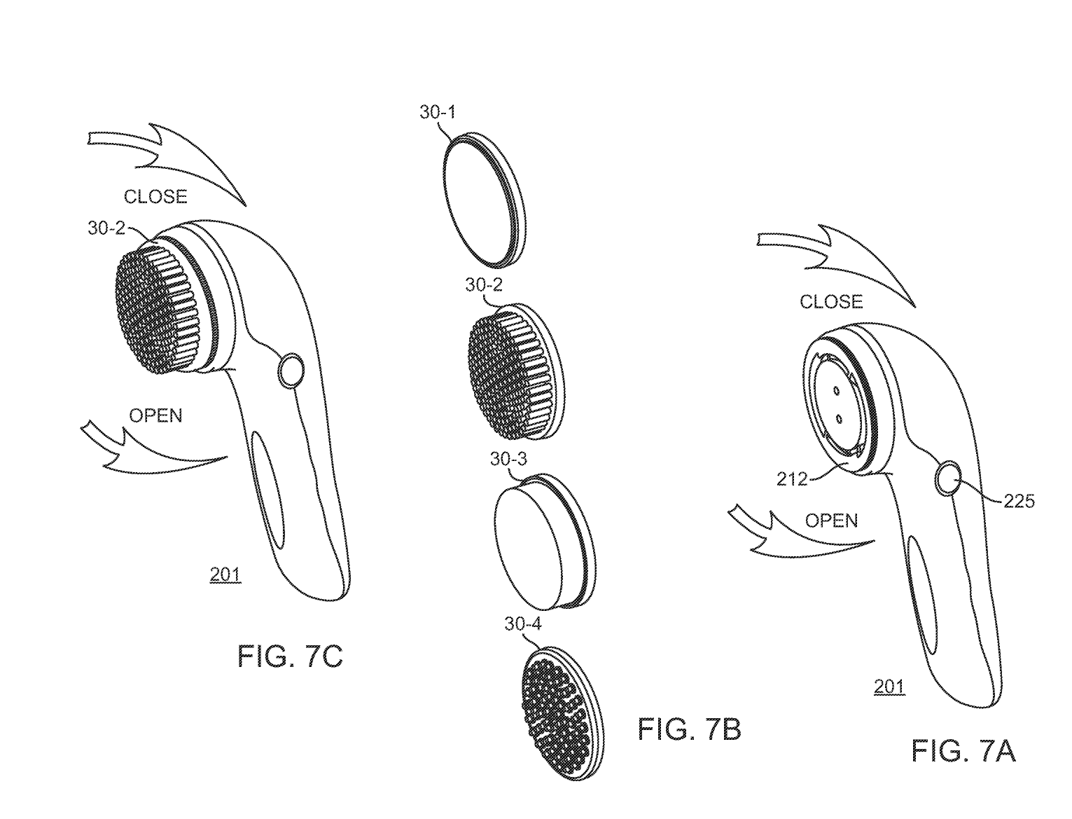

In one embodiment of the device 201, the attachment head holder 212 accepts an attachment head 214 having a contact surface for skin contact (FIG. 5). FIG. 7A shows a perspective view of the device 201, and FIG. 7B shows a different example of the attachment head 214 designated as attachment heads 30-1, 30-2, 30-3 and 30-4. The attachment head holder 212 accepts different attachment heads using a twist and lock operation in the same manner as the attachment head holder 12 described above for the device 101. Further, attachment heads 30-1, 30-2, 30-3 and 30-4 are similar to the attachment heads 29-1, 29-2, 29-3 and 29-4, respectively, described above. FIG. 7C shows the device 201 with the attachment head 30-2 engaged in the holder 212. The attachment heads 30-1, 30-2, 30-3 and 30-4 may be coupled to the attachment head holder 212 in the same manner that was described above for coupling the attachment heads 29-1, 29-2, 29-3 and 29-4 to the attachment head holder 12. The attachment heads 30-1, 30-2, 30-3 and 30-4 may be decoupled from the holder 212 using a twist and unlock motion, similar to that described for device 101.

The device 201 includes a recharging circuit 205 on a printed circuit board (PCB) for charging the rechargeable batteries 227. The recharging circuit 205 is connected to a DC input jack 202 for receive DC power from an external source. The recharging circuit 205 may be of a well-known conventional type. A charger pin 203 provides recharging power to the rechargeable batteries 227.

The device 201 further includes a main PCB 221 which includes electronic circuitry for controlling operation of the motor 223, in one example includes circuitry for controlling the motor to operate with 20 seconds clockwise and anti-clockwise intervals, and maintains a steady voltage for the timer.

The power DC jack 202 may also provide power to rechargeable charge batteries 227 and/or operate the electric motor 223 directly from an external AC-DC adapter. The external AC-DC adapter may be a conventional adapter such as 120V AC to 9V DC adaptor. When the rechargeable batteries 227 have sufficient charge, the device 201 may be operated as a handheld cordless unit that is powered by the batteries therein. In one example, when the device 201 is connected to an external AC-DC adapter via DC jack 202, the PCB circuit 221 automatically switches between operation from external power from the DC jack 202 to battery operation by sensing the sufficiency of battery charge for operation of the motor 223.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

The corresponding structures, materials, acts, and equivalents of all means or step plus function elements in any claim are intended to include any structure, material, or act for performing the function in combination with other claimed elements. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. An automated hair removal system, comprising:
a handheld device comprising an electric motor, an attachment holder and an attachment head;
the electric motor for rotating the attachment holder, wherein the attachment holder comprises a head attachment mechanism for receiving the attachment head;
the attachment head comprising an abrasive contact surface configured for skin contact, and a holder attachment mechanism for detachably attaching the attachment head to the attachment holder;
wherein rotation of the attachment holder by the electric motor causes rotation of the attachment head and the contact surface, and wherein rotation of the contact surface upon placement against the skin essentially in parallel to the skin and transverse to hair thereon, removes hair from the skin by tapering and weakening the structure of the hair.

2. The system of claim 1, wherein the handheld device further comprises:
a housing having a handle portion and a head portion, wherein the head portion contains said electric motor and has an opening for coupling said attachment holder to the electric motor therethrough; and
plural interlocking transfer gears disposed in the head portion, said interlocking gears coupling a shaft of the motor to the attachment holder through said opening, the interlocking transfer gears transferring rotation of said shaft to the attachment head holder.

3. The system of claim 2, wherein the transfer gears comprise reduction gears for successively reducing rotation transferred from the motor shaft to the attachment holder.

4. The system of claim 3, wherein the attachment holder comprises an essentially circular member having a pair of opposing faces, said head attachment mechanism being disposed on a first face of the circular member, and the attachment holder being coupled to a transfer gear at an opposing face of the circular member.

5. The system of claim 4, wherein rotation of the attachment holder and the attachment head is in a plane essentially perpendicular to an axis of rotation of the motor shaft.

6. The system of claim 2, wherein the handle portion includes a cavity for disposing an electrical power source for the electric motor.

7. The system of claim 5, wherein the attachment head comprises an essentially disc member having a pair of opposing faces, said holder attachment mechanism being disposed on a first face of the disc member, and the contact surface being disposed at an opposing face of the disc member.

8. The system of claim 7, wherein:
the head attachment mechanism of the attachment holder comprises a pair of curved channels on said first face of the circular member;
the holder attachment mechanism of the attachment head comprises a pair of essentially L-shaped protrusions on said first face of the disc member, such that the channels are shaped to receive and releasably engage the L-shaped protrusions to securely maintain the attachment head on the attachment holder.

9. The system of claim 7, wherein:
the head attachment mechanism of the attachment holder comprises a first opening and a second opening on said first face of the circular member;
the holder attachment mechanism of the attachment head comprises an L-shaped protrusion, and at least a flexing spring tab transverse to said first face of the disc member, such that the openings are shaped to receive and releasably engage the L-shaped protrusion and the flexing spring tab, to securely maintain the attachment head on the attachment holder.

10. The system of claim 9, wherein:
the flexing spring tab is bendable to allow releasably clipping the attachment head to the attachment holder by holding the first face of the attachment head against the first face of the attachment holder, placing the flexing spring tab in said first opening of the attachment holder, and pushing the attachment head against the attachment holder causing the flexing spring tab to bend until the L-shaped protrusion snaps into said second opening of the attachment holder, whereby the attachment head is held in place as the flexing spring tab urges back into said first opening and the L-shaped protrusion engages said second opening.

11. The system of claim 9, wherein said contact surface comprises an abrasive sand paper disc for removing hair from the skin by tapering and weakening the structure of the hair.

12. An automated skin conditioning system, comprising:
a handheld device comprising an electric motor, an attachment holder and an attachment head;
the electric motor for rotating the attachment holder, wherein the attachment holder comprises a head attachment mechanism for receiving the attachment head; and
the attachment head comprising a contact surface configured for skin contact, and a holder attachment mechanism for detachably attaching the attachment head to the attachment holder, allowing coupling of different attachment heads to the attachment holder;
wherein rotation of the attachment holder by the electric motor causes rotation of the attachment head and the contact surface, wherein rotation of the contact surface upon placement against the skin essentially in parallel to the skin.

13. The system of claim 12, wherein the handheld device further comprises:
a housing having a handle portion and a head portion, wherein the head portion contains said electric motor and has an opening for coupling said attachment holder to the electric motor therethrough; and
plural interlocking transfer gears disposed in the head portion, said interlocking gears coupling a shaft of the motor to the attachment holder through said opening, the interlocking transfer gears transferring rotation of said shaft to the attachment holder, wherein the transfer gears comprise reduction gears for successively reducing rotation transferred from the motor shaft to the attachment holder;
wherein the attachment holder comprises an essentially circular member having a pair of opposing faces, said head attachment mechanism being disposed on a first face of the circular member, and the attachment holder being coupled to a transfer gear at an opposing face of the circular member,
wherein the attachment head comprises an essentially disc member having a pair of opposing faces, said holder attachment mechanism being disposed on a first face of the disc member, and the contact surface being disposed at an opposing face of the disc member;
such that rotation of the attachment holder and the attachment head is in a plane essentially perpendicular to an axis of rotation of the motor shaft.

14. The system of claim 13, wherein:
the head attachment mechanism of the attachment holder comprises a first opening and a second opening on said first face of the circular member;
the holder attachment mechanism of the attachment head comprises an L-shaped protrusion, and at least a flexing spring tab transverse to said first face of the disc member, such that the openings are shaped to receive and releasably engage the L-shaped protrusion and the flexing spring tab, to securely maintain the attachment head on the attachment holder;
such that the flexing spring tab is bendable to allow releasably clipping the attachment head to the attachment holder by holding the first face of the attachment head against the first face of the attachment holder, placing the flexing spring tab in said first opening of the attachment holder, and pushing the attachment head against the attachment holder causing the flexing spring tab to bend until the L-shaped protrusion snaps into said second opening of the attachment holder, whereby the attachment head is held in place as the flexing spring tab urges back into said first opening and the L-shaped protrusion engages said second opening.

15. The system of claim 13, wherein said contact surface comprises one of: a brush head contact disc, a sponge head contact disc, a massage head contact disc having rubber massaging nubs, and an abrasive sand paper disc for removing hair from the skin by tapering and weakening the structure of the hair.

16. The system of claim 13, wherein the handheld device further comprises a rechargeable power source with input terminals, and the system further comprises a charging base for receiving the handheld device, the charging base including output terminals for making electrical contact with the input terminals of the power source for charging the power source.

17. A method of removing hair from skin, comprising:
providing an automated hair removal system including:
a handheld device comprising an electric motor, an attachment holder and an attachment head;
the electric motor for rotating the attachment holder, wherein the attachment holder comprises a head attachment mechanism for receiving the attachment head;
the attachment head comprising an abrasive contact surface configured for skin contact, and a holder attachment mechanism for detachably attaching the attachment head to the attachment holder;
wherein rotation of the attachment holder by the electric motor causes rotation of the attachment head and the contact surface;
lightly pressing the contact surface on an area of the skin and passing the rotating contact surface over the skin, wherein rotation of the contact surface upon placement against the skin essentially in parallel to the skin and transverse to hair thereon, removes hair from the skin by tapering and weakening the structure of the hair.

18. The method of claim 17 further comprising repeatedly passing the rotating contact surface over an area of the skin with hair, wherein with repeated application, hair becomes thinner and finer and hair removal intervals become longer.

19. The method of claim 17, wherein the handheld device further comprises:
a housing having a handle portion and a head portion, wherein the head portion contains said electric motor and has an opening for coupling said attachment holder to the electric motor therethrough; and
plural interlocking transfer gears disposed in the head portion, said interlocking gears coupling a shaft of the motor to the attachment holder through said opening, the interlocking transfer gears transferring rotation of said shaft to the attachment holder, wherein the transfer gears comprise reduction gears for successively reducing rotation transferred from the motor shaft to the attachment holder;
wherein the attachment holder comprises an essentially circular member having a pair of opposing faces, said head attachment mechanism being disposed on a first face of the circular member, and the attachment holder being coupled to a transfer gear at an opposing face of the circular member;
wherein the attachment head comprises an essentially disc member having a pair of opposing faces, said holder attachment mechanism being disposed on a first face of the disc member, and the contact surface being disposed at an opposing face of the disc member;
such that rotation of the attachment holder and the attachment head is in a plane essentially perpendicular to an axis of rotation of the motor shaft.

20. The method of claim 19, wherein:
the head attachment mechanism of the attachment holder comprises a first opening and a second opening on said first face of the circular member;
the holder attachment mechanism of the attachment head comprises an L-shaped protrusion, and at least a flexing spring tab transverse to said first face of the disc member, such that the openings are shaped to receive and releasably engage the L-shaped protrusion and the flexing spring tab, to securely maintain the attachment head on the attachment holder;
such that the flexing spring tab is bendable to allow releasably clipping the attachment head to the attachment holder by holding the first face of the attachment head against the first face of the attachment holder, placing the flexing spring tab in said first opening of the attachment holder, and pushing the attachment head against the attachment holder causing the flexing spring tab to bend until the L-shaped protrusion snaps into said second opening of the attachment holder, whereby the attachment head is held in place as the flexing spring tab urges back into said first opening and the L-shaped protrusion engages said second opening.
